(19) United States
(12) Patent Application Publication
(10) Pub. No.: US 2021/0058736 A1
(43) Pub. Date: Feb. 25, 2021

(54) PROXIMITY ALERT AND CONTACT TRACING DEVICE, METHOD AND SYSTEM

(71) Applicant: OTTOGEE, INC, Dallas, TX (US)

(72) Inventors: Omar Ghazzaoui, Dallas, TX (US); Jonathan Taylor, Dallas, TX (US)

(21) Appl. No.: 16/844,898

(22) Filed: Apr. 9, 2020

Related U.S. Application Data
(63) Continuation-in-part of application No. 29/683,010, filed on Mar. 10, 2019.

(51) Int. Cl. H04W 4/02; H04W 4/029; H04W 4/80; H04W 4/90; G06K 19/07; G08B 21/02; G08B 21/22; G08B 7/06; G16H 50/80

(52) U.S. Cl. CPC H04W 4/023; H04W 4/029; H04W 4/80; H04W 4/90; G16H 50/80; G08B 21/02; G08B 21/22; G08B 7/06; G06K 19/0723

(57) ABSTRACT

A system, method and device for tracing contact or proximity between people and between people and items to be tracked, wherein data is recorded by the device and contacts between people and other people or things is traced for disease transmission containment or for optimized asset utilization and fraud prevention. In a preferred embodiment, the device communicates stored data to a second device. In a further preferred embodiment, when a proximity threshold has been violated between two devices that have come too close to each other, an alarm in each device is activated to alert the persons bearing the device that a proximity violation has occurred, such that the proximity violation is immediately corrected by the individuals involved moving apart so that the alarm returns to a deactivated state. The device, system and method of this invention provides a valuable readiness resource for pandemic disease spread containment.

This application Claims Priority to U.S. Ser. No. 29/683,010, filed on Mar. 10, 2019, the contents of which are herein incorporated by reference

### 1.0 FIELD OF THE INVENTION

A system, method and device for tracing contact or proximity between people and between people and items to be tracked, including for pandemic disaster readiness and containment.

### 2.0 BACKGROUND OF THE INVENTION

In a world with ever increasing numbers of people, there are many instances in which it is desirable, if possible, to identify when, where, for how long and to what extent people have come into contact with each other or with a thing, meaning any device, equipment, or even animals. This invention has multiple applications, including provision of asset tracking, risk reduction, and acuity sufficient to provide information on who was in proximity with whom or with what, where, when, and for how long.

In 2020, the world has experienced a modern pandemic as a result of a coronavirus outbreak, which has caused the disease entitled COVID-19, the spread of which is tightly correlated to proximity between non-infected people and infected people, animals, or fomites, namely, anything that an infected person has had contact with and left disease containing residue on, contact with any of which being sufficient to cause an individual to contract the disease. In addition, the Internet of Things (“IOT”) has made it possible to locate a wide variety of devices which are internet or Bluetooth® or otherwise communication enabled Worldwide, wireless communication has become standard. Of particular import to the present invention are communication technologies which operate over relatively short distance ranges, i.e. proximity, from direct contact to several feet or meters.

Bluetooth® technology is set to be embedded in close to 10 billion devices in 2020. Operating in the 2.4 GHz frequency band across 79 channels, it is most common in cell phones but also designed to operate wireless PC and audio equipment and hands-free headsets, plus allow digital downloads between devices and a connection with the interface of vehicles. As such, any two Bluetooth-®-compatible devices can send and receive data wirelessly and without any reliance on Wi-Fi. Bluetooth® V2.1 had a range of up 100 meters, as did 4.0 (LE). The latest version, Bluetooth® 5, has increased that range to a possible 400 meters. Two benefits of short-range frequencies are low power consumption and the fact that they are (relatively) secure. Both Bluetooth®'s Low Energy (“BLE”) and Basic Rate/Enhanced Data Rate iterations offer multi-channel, spread spectrum (commonly called “channel hopping”) solutions. This ensures a private frequency is always available to users, with security robust enough to be classified as “government grade.” Furthermore, Bluetooth® is a versatile and developer-friendly platform. In addition, higher-powered transmitters can increase the effective range of Bluetooth® more than ten times.

RFID stands for Radio Frequency Identification. It consists of a chip and antenna embedded in an object that can be scanned, identified, and tracked via radio waves. Like Bluetooth®, RFID is capable of both transmission and reception via antenna and microchip processor. Tags can be mass-produced cheaply, though the readers tend to be expensive. The data stored on RFID tags is item-specific and thus can be used to differentiate between unique tags in other devices. For example, if a shipment of 100 identical toys is displayed in a store, staff can identify each one individually by the serial number on its RFID chip. This is also the method used when a family pet is chipped under the skin for later identification. RFID operates under a global standard which improves efficiency and security. The tags are classified under 6 types (0 to 5). Types of chips range from passive to active and can allow the tags to communicate with each other, transfer power between themselves, and record factors like motion, temperature, and pressure. Unfortunately, RFID's dependence on Wi-Fi makes it easier to disrupt than Bluetooth®. RFID tags also have trouble responding to simultaneous scanner queries and may suffer when many tags are present in a small area. When it's used for inventory purposes, tags remain active after leaving the supply chain, which renders them vulnerable to being scanned again. For example, vendors or even criminals could use still-active RFID chips to scan the contents of shopping bags and base a sales pitch or a theft on the information they gain.

NFC (Near Field Communication) is akin to both RFID and Bluetooth® in that it is an evolution of the former and, similar to Bluetooth®, it lets mutually-NFC-compatible devices communicate and exchange various forms of data. It has the shortest send and receive range of all three technologies at only a matter of inches. Again, like Bluetooth®, this shorter transmission range means more effective security, and there are instances in which the technology of the present invention may implement NFC communication for detailed proximity acuity. NFC is being used by companies like Samsung, Android, and Apple to allow customers to pay for their purchases. It has an advantage over Bluetooth® in that NFC devices don't need to be specifically paired to interact; the disadvantage is that NFC is slower in transmission and reception. NFC is operable across three modes: read/write, card emulation, and peer-to-peer. Read/write can be used in advertising for promotional offers, such as scanning an icon in a magazine, billboard, or poster to get more information on an offer. Card emulation allows NFC devices to act like credit cards, while peer-to-peer allows data sharing between two users. This method of data transfer is set to take off in a major way. It can be embedded in everything from parking meters and wearable items to tattoos or sub-dermal implants. NFC also uses less energy than Bluetooth®, with its passive components requiring no power supply. It is NFC's non-paired connectivity and its use in making payments that could make it the most commonly utilized of the three technologies. It is the perfect medium to pay for and receive things quickly—and this convenience is a high priority for consumers as well as merchants.

Accordingly, one significant application of this invention is in providing data, including in some embodiments, real-time or as much a real-time data as possible, as well as retrospective data, is secured utilizing near-proximity technologies available today such as, but not limited to, Bluetooth®, RFID or NFC, to assist, in one embodiment, in tracking and controlling the spread of a pathogen or disease by tracking and controlling the movements and contacts of infected individuals, and in another embodiment, tracking and controlling the movements, proximity, contacts, and duration of contacts of people in relation to other assets.

The present invention provides an inexpensive, dedicated and important solution to assist in combatting the present and future pandemics, acts of bioterrorism, and the like. The system is an essential tool to slow down the spread of infectious disease through contact tracing, where the channels a disease is spreading through can be identified and halted. The device, system and method of this invention makes it possible to locate and isolate infected individuals and those that have come into contact with them without requiring the dedication of hundreds of workhours by healthcare professionals. In addition, the system, method and device according to the present invention likewise permits optimization of use of assets, risk avoidance (e.g. the wrong person is in the wrong place or at the wrong time or is in proximity to the wrong device or is using the asset for too long, or the like), with significant ability to avoid accidents when the system issues an alert of an improper location, contact, proximity, time, or duration of proximity/contact.

### 3.0 BRIEF SUMMARY OF THE INVENTION

In one embodiment according to this invention, the device, system and method comprises a simple Bluetooth® enabled device, an NFC enabled device, or a radiofrequency enabled device, (such as an RFID tag), or the like, in the form of but not limited to, a bracelet, a pin, a card, or any other portable device, which detects other similarly enabled devices, such as bracelets, pins or the like that are within a given proximity range, say two meters or less from each other. Through proximity sensing technology known in the art, the system traces such contact events or proximity events and stores the data for real time or retrospective analysis. In a preferred version of this embodiment, when two people are in such proximity or contact so as to violate the proximity threshold coded into the device, the device emits an alarm, which may be audible, visual, vibratory, or the like and combinations thereof, on one or both of the devices. Accordingly, in such an embodiment, a self-contained solution requiring no external resources, databases, computers, uplinks delay times, or expensive secondary equipment such as cell phones or the like, nonetheless provides in important tool to prevent prohibited contacts by alerting the device-bearing individuals to regain accepted distancing from each other. Sufficiently broadly distributed, at low cost (e.g. well below $100 per device), large segments of the world's population would be better equipped for pandemic disaster readiness.

In another embodiment, the device further comprises a wi-fi transponder to provide acuity on precise location all in one device in yet a further embodiment, the device transmits data, via wi-fi, Bluetooth®, or any other means to a secondary device, such as a cell phone, which has cellular, wi-fi, Bluetooth® or a combination of such capabilities for transmission of the data to health care professionals, asset managers or the like to enable warnings or corrective actions to be taken to prevent spread of infection or inappropriate use of tracked assets.

Contact and proximity data, along with time and duration, are recorded by the device for every contact or proximity detected within pre-set limits. The data is optionally encrypted and stored on the device for as long as needed, and the data from the device may be transmitted, uploaded or downloaded to another device, such as a cell phone, a computer, or the like for loading into analytic software on the computer or on the internet including in the cloud, for example.

In an embodiment comprising a Wi-Fi transponder, cellular correspondence hardware, global positioning technology, or combinations thereof, either all on board on the device or via communication with another device, such as a cell phone, data comprising time, location, duration and identity of the individuals or other assets involved in the proximity events are uploaded to the cloud, immediately as acquired, or pursuant to pre-set delays, for subsequent or real-time analytics for prospective action if need, e.g. isolation of infected individuals and those they have come into close and prolonged contact with; intervention with. The data on each device, e.g. bracelet reveals who each person wearing the bracelet is, and who they interacted with, when, where and for how long.

This invention provides a solution to a long felt need, and may be deployed in the general public, or, more easily and initially, in more contained populations and locations, such as in workforces, security or armed forces, small political circles, in senior care facilities, hospitals, workspaces, or any other relatively contained communities in which each member of the community wears a device which participates in the system described herein to track proximity or contacts between individuals and between individuals and things. In due course, as the system is deployed broadly, more and more contact data between people and things equipped to communicate with each other is obtained, providing greater precision and acuity.

### 4.0 BRIEF DESCRIPTION OF THE FIGURES

FIG. 1 provides an overview of the operation of the device, system and method of this invention as applied to preventing disease transmission.

FIG. 2A-2C provide exemplary screenshots to show the user interface for using the device, system and method according to this invention to prevent disease transmission.

FIGS. 3A-3B provide additional exemplary screenshots to show the interaction of a bracelet embodiment of the device in the process of uploading data to a mobile phone running the application providing the user interfaces shown.

FIG. 4 shows intervention by the system to notify at risk individuals that they may need medical attention or the like due to contact with an infected individual.

FIG. 5A provides a dashboard showing tracking of workers on a worksite where each worker carries a device according to this invention to provide real-time tracing of their locations.

FIG. 5B shows an embodiment of the dashboard in which artificial intelligence (AI) is implemented to predict safety events before they occur.

FIG. 6A provides a dashboard showing tracking of equipment on a worksite where each piece of equipment is tagged with a device according to this invention to provide real-time tracing of the asset's location.

FIG. 6B provides a graphic summary of various aspects of the device, system and method of this invention whereby AI is implemented to collect and analyze data from all sources, including enterprise assets, heavy equipment, power tools, environmental sensors, materials, and the like to provide real-time insights, automation of routine administrative tasks, anonymized data when needed, qualified performance, AI optimized algorithms, identified skill gaps and measures of motivation. The assets are preferably enabled with one or more communication enabled devices such cellular enabled IoT hardware, to leverage next generation IoT infrastructure, Bluetooth® enabled for device to device and device communications and location tracking, GPS enabled for precise outdoor positioning, WiFi enabled for indoor positioning, all connected to an expandable sensor platform for application specific functionality. Preferably, BLE is used to achieve Low-Power Optimization.

FIG. 7 provides a graphic depiction of one embodiment of a device according to this invention in the form of a smart badge holder which includes cellular, Bluetooth®, WiFi, and GPS all in one compact device for use in the system according to this invention, to connect resources using safe, secure and un-intrusive IoT-enabled wearables and low-power networked sensors, data analytics for resource allocation, utilization, performance, efficiency, skill gaps and the like, enabling rapid, fact-based decision-making through real-time analysis, and synthesis of data to achieve desired results while improving the working environment of the workforce.

FIG. 8 provides a tabular representation of features of the system providing a comprehensive IoT solution that transforms workforce management across six dimensions.

FIG. 9A provides a top view diagrammatic representation of the ACTIVE CARD HOLDER showing a base housing containing active electronics and card retainer ridges which retain a card in association with said base housing when an interchangeable identification card is situated between the card retainer ridges and the top side of said base; 9B provides a top perspective diagrammatic representation of the ACTIVE CARD HOLDER showing a charging port interconnected with active electronics contained within the ACTIVE CARD HOLDER base, also showing the card retainer ridges which retain an interchangeable card in association with the base when the interchangeable card is in position between the card retainer ridges and the base housing; 9C provides an end-on diagrammatic representation of the ACTIVE CARD HOLDER viewed with the top of the base, containing active electronics, flipped downward as compared to the orientation provided in FIG. 9A, showing the base and card retainer ridges in end-on profile; FIG. 9D provides a side diagrammatic representation of the ACTIVE CARD HOLDER viewed in the same orientation as in FIG. 9C, but rotated 90 degrees to show the side view rather than an end-on view of the active card holder, viewed with the base, containing active electronics, with the top thereof flipped downward as compared to FIG. 9A, showing the base and card retainer ridges in side profile view with a power switch and cable ports. The active card reader provides one embodiment of a device according to this invention which is included in a system according to this invention for use according to the method disclosed herein. For purposes of this aspect of the invention, priority is herein claimed to prior co-pending filing U.S. Ser. No. 29/683,010, filed on Mar. 10, 2019.

FIG. 10 provides a top diagrammatic representation of the ACTIVE CARD HOLDER viewed from above with an interchangeable identification card in place, (FIGS. 10A and 10B), retained in association with the active base by the card retainer ridges, wherein FIG. 10A, shows a rectangular base in which not all four sides are substantially equal in length, while FIGS. 10B, 10C and 10D show a rectangular base in which all four sides are substantially equal in length.

FIG. 11 provides a representation of data captured according to the system of this invention in which daily contact metrics are captured, including the total number of contacts captured, the number of contacts per day, the duration of contacts and the average contact duration, as well as a contact trace graph.

FIG. 12 provides a representation of data captured according to the system of this invention in which daily contact metrics are captured, including the total number of contacts captured, the number of contacts per day, the duration of contacts and the average contact duration, along with daily test metrics.

FIG. 13 provides a representation of data captured according to the system of this invention in which daily contact metrics are captured, including the total number of contacts captured, the number of contacts per day, the duration of contacts and the average contact duration, along with a table showing contacts.

### 5.0 DETAILED DISCLOSURE OF THE PREFERRED EMBODIMENTS ACCORDING TO THE INVENTION

The present system, method and device in one embodiment according to this invention, comprises short range communication hardware and software enabling Bluetooth® or other forms or radiofrequency communication, such as an RFID, NFC, or the like, in the form of a bracelet, a pin, a card, or any other portable device, which detects other similarly enabled devices, such as bracelets, pins, cards, tags, or the like that are within a given range of say two meters or less from each other Data is optionally encrypted and stored on the device for as long as needed, and the data from the device may be downloaded/uploaded to another device, such as a computer or a phone or a dedicated gateway for loading into analytic software on the computer or on the internet (in the cloud, for example), or, in one embodiment, the device is wi-fi enabled and uploads data to the cloud immediately as acquired for real-time analytics and prospective action if needed, e.g. isolation of infected individuals and those they have come into close and prolonged contact with infected individuals, or intervention to prevent a given individual from having improper access to or contact with a given trackable asset. The data on each device, e.g. bracelet reveals who each person wearing the bracelet is, and who or what they interacted with, when, where, and for how long.

In one primary embodiment, the device according to this invention comprises a housing for a wireless communication system to provide real-world, significant outcome changes. Of particular relevance to the present invention is a device enabled with known short-range wireless communication technologies, including but not limited to Bluetooth®, NFC, and RFID.

In a preferred embodiment, Bluetooth® is used in the device, system and method of this invention, as Bluetooth® is a wireless standard designed to replace data cables while providing two-way communication options within a range of about 10 meters. Bluetooth® is built into most mobile phones and many consumer electronics devices.

In another preferred embodiment, the device is NFC (Near Field Communication) enabled, as NFC tags can be used for virtually unlimited applications and all it takes to read them is a regular NFC-enabled device. Like Bluetooth®, NFC supports two-way communication between devices and is built into over 1 billion devices, including smartphones and a growing number of tablets, PCs, gaming consoles, consumer electronics devices, and household appliances. However, for greater security and control, NFC works within a close range of a couple of inches.

Accordingly, the device according to the present invention incorporates a wireless communication transponder, which senses and records proximity of like transponders or tags on people or other assets.

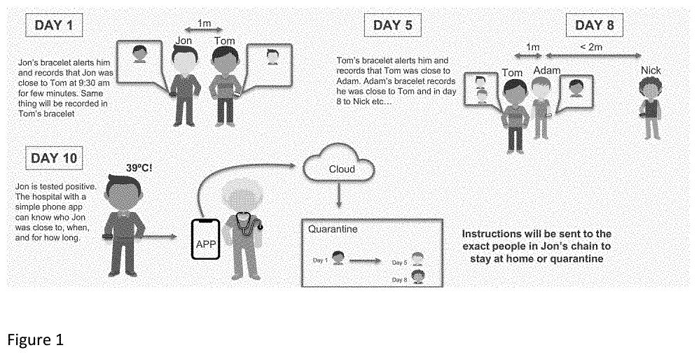

In FIG. 1, there is provided an overview of one embodiment of the component devices and methods relevant to implementation of the device, system and method of this invention as applied to preventing disease transmission.

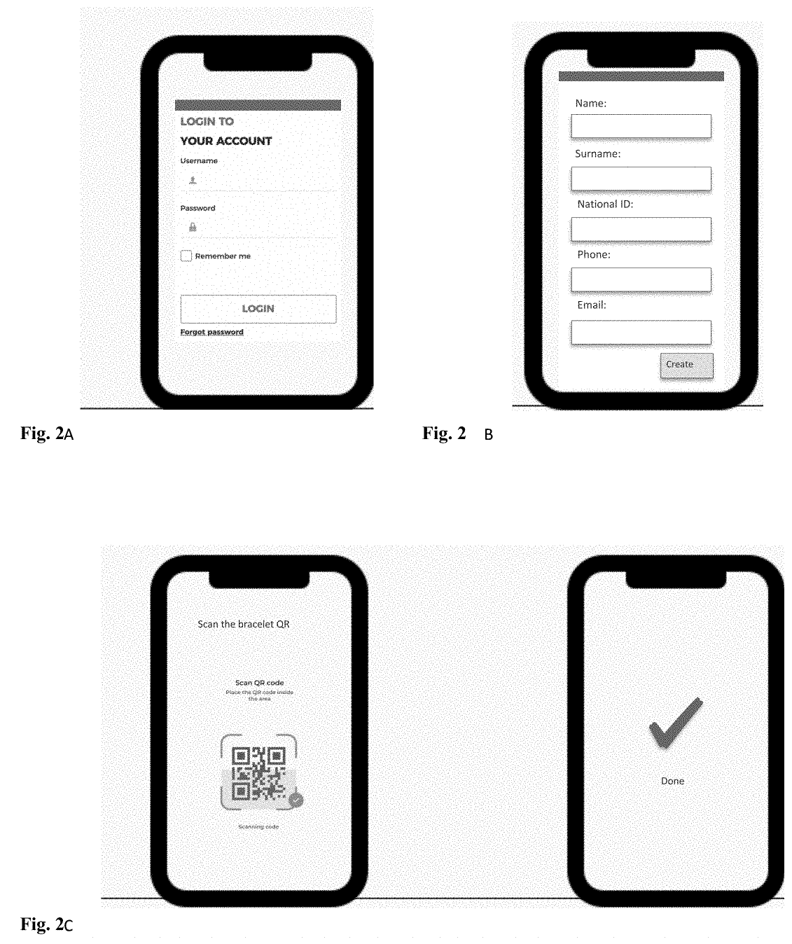

FIG. 2A-2C provide exemplary screenshots to show the user interface for using the device, system, and method according to this invention to prevent disease transmission.

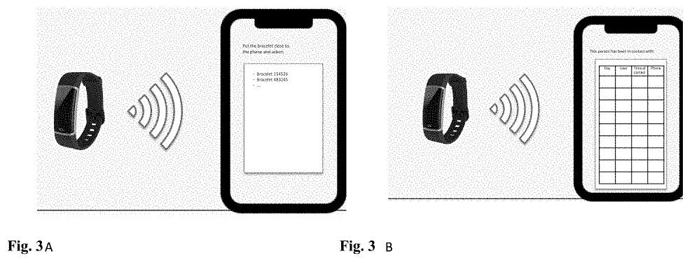

FIGS. 3A-3B provide additional exemplary screenshots to show the interaction of a bracelet embodiment of the device in the process of uploading data to a mobile phone running the application providing the user interfaces shown.

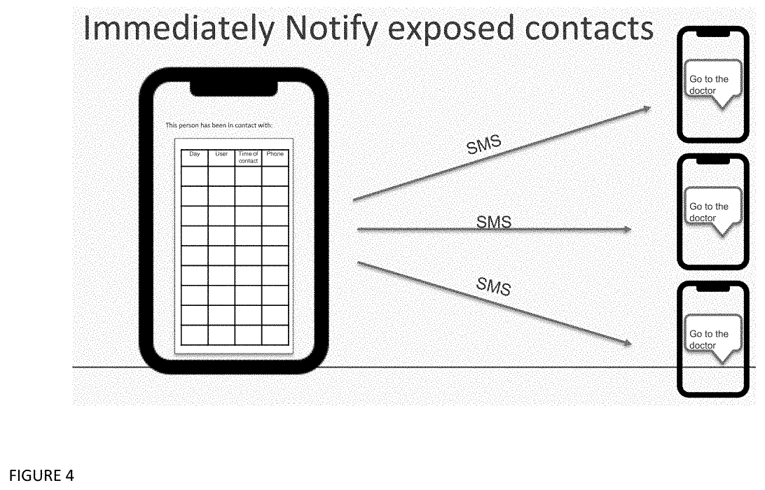

FIG. 4 shows intervention by the system to notify at risk individuals that they may need medical attention or the like due to contact with an infected individual.

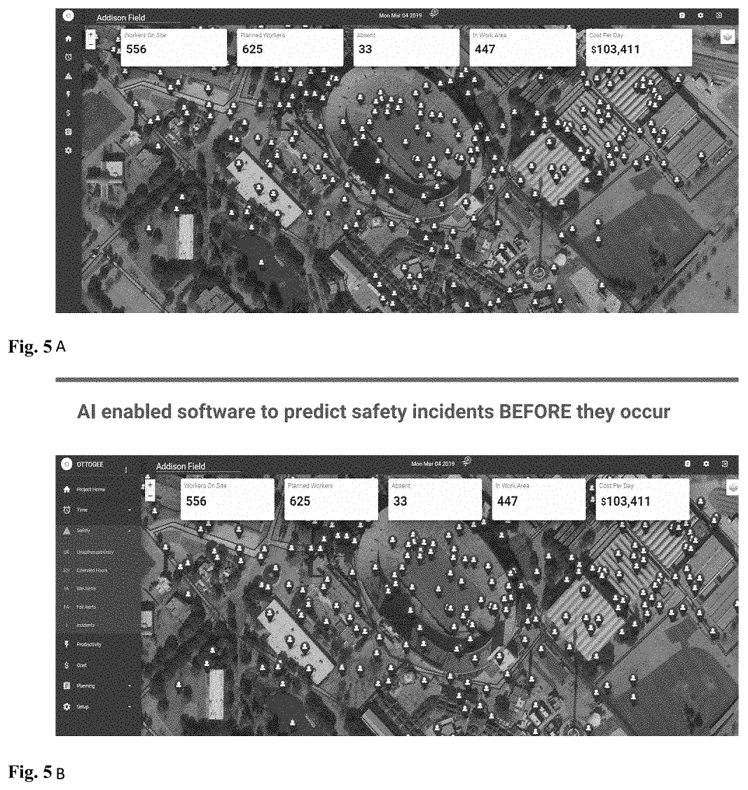

FIG. 5A provides a dashboard showing tracking of workers on a worksite where each worker carries a device according to this invention to provide real-time tracing of their locations.

FIG. 5B shows an embodiment of the dashboard in which artificial intelligence (AI) is implemented to predict safety events before they occur.

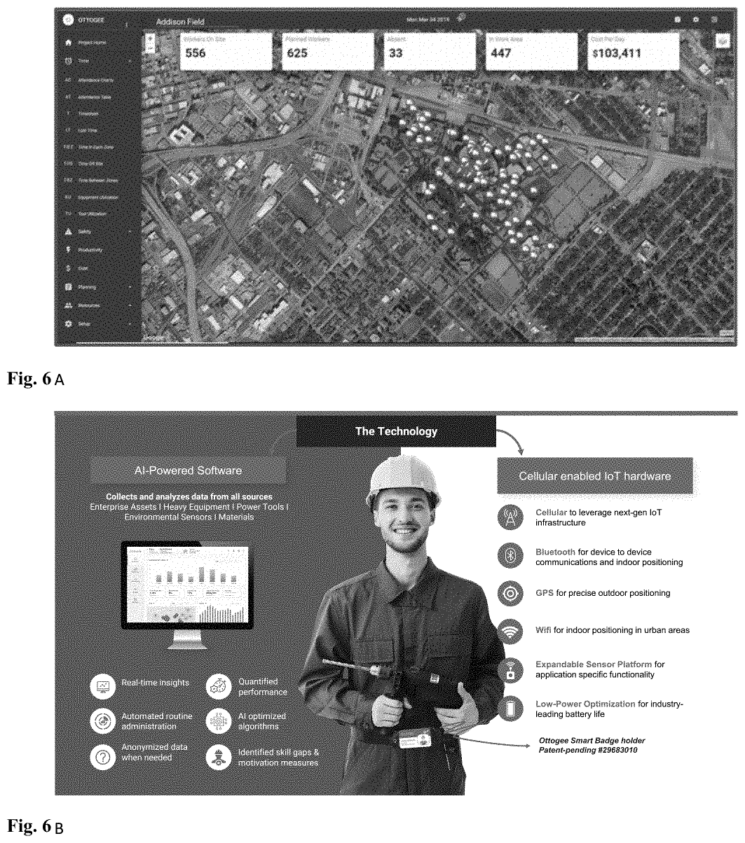

FIG. 6A provides a dashboard showing tracking of equipment on a worksite where each piece of equipment is tagged with a device according to this invention to provide real-time tracing of the asset's location.

FIG. 6B provides a graphic summary of various aspects of the device, system and method of this invention whereby AI is implemented to collect and analyze data from all sources, including enterprise assets, heavy equipment, power tools, environmental sensors, materials, and the like to provide real-time insights, automation of routine administrative tasks, anonymized data when needed, qualified performance, AI optimized algorithms, identified skill gaps and measures of motivation. The assets are preferably enabled with one or more communication enabled devices such cellular enabled IoT hardware, to leverage next generation IoT infrastructure, Bluetooth® enabled for device to device and device communications and location tracking, GPS enabled for precise outdoor positioning, WiFi enabled for indoor positioning, all connected to an expandable sensor platform for application specific functionality. Preferably, BLE is used to achieve Low-Power Optimization.

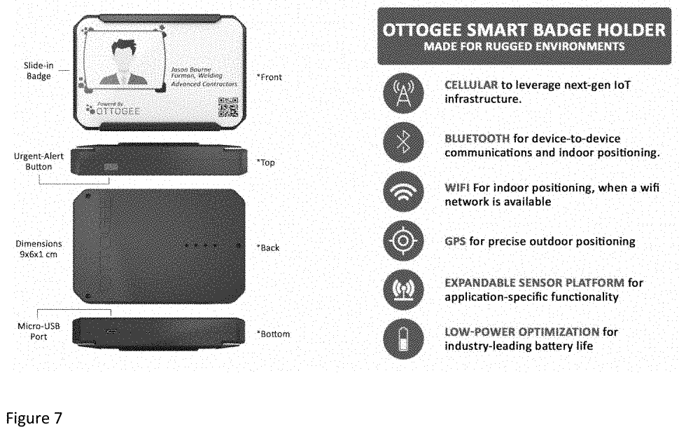

FIG. 7 provides a graphic depiction of one embodiment of a device according to this invention in the form of a smart badge holder which includes cellular, Bluetooth®, WiFi, and GPS all in one compact device for use in the system according to this invention, to connect resources using safe, secure and un-intrusive IoT-enabled wearables and low-power networked sensors, data analytics for resource allocation, utilization, performance, efficiency, skill gaps and the like, enabling rapid, fact-based decision-making through real-time analysis, and synthesis of data to achieve desired results while improving the working environment of the workforce.

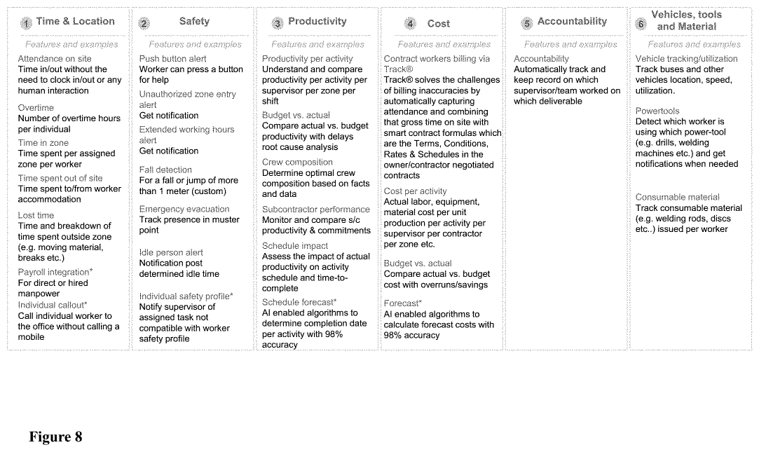

FIG. 8 provides a tabular representation of features of the system providing a comprehensive IoT solution that transforms workforce management across six dimensions.

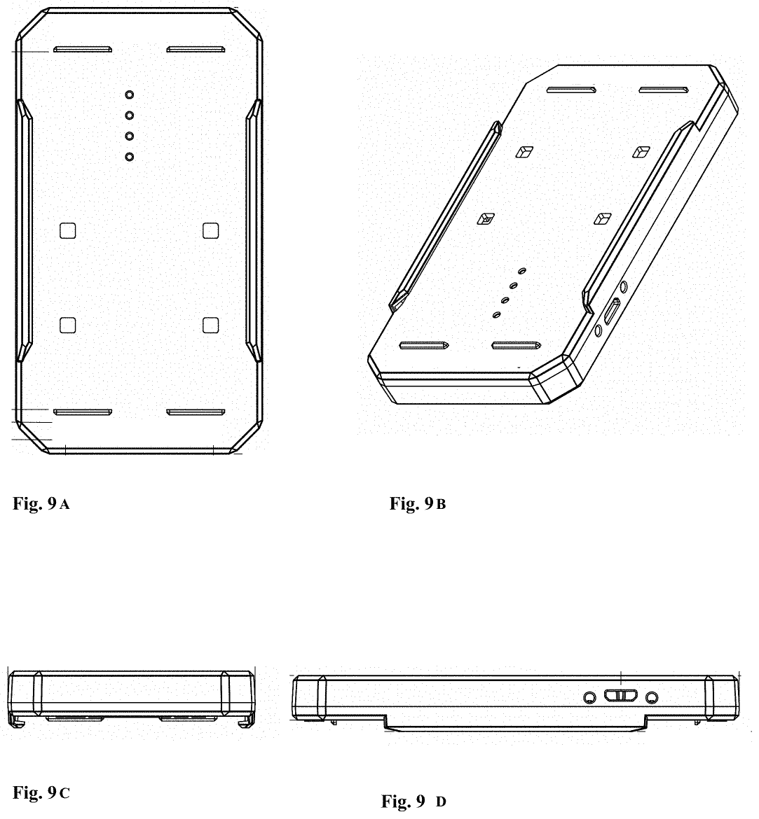

FIG. 9A provides a top view diagrammatic representation of the ACTIVE CARD HOLDER showing a base housing containing active electronics and card retainer ridges which retain a card in association with said base housing when an interchangeable identification card is situated between the card retainer ridges and the top side of said base; 9B provides a top perspective diagrammatic representation of the ACTIVE CARD HOLDER showing a charging port interconnected with active electronics contained within the ACTIVE CARD HOLDER base, also showing the card retainer ridges which retain an interchangeable card in association with the base when the interchangeable card is in position between the card retainer ridges and the base housing; 9C provides an end-on diagrammatic representation of the ACTIVE CARD HOLDER viewed with the top of the base, containing active electronics, flipped downward as compared to the orientation provided in FIG. 9A, showing the base and card retainer ridges in end-on profile;

FIG. 9D provides a side diagrammatic representation of the ACTIVE CARD HOLDER viewed in the same orientation as in FIG. 9C, but rotated 90 degrees to show the side view rather than an end-on view of the active card holder, viewed with the base, containing active electronics, with the top thereof flipped downward as compared to FIG. 9A, showing the base and card retainer ridges in side profile view with a power switch and cable ports.

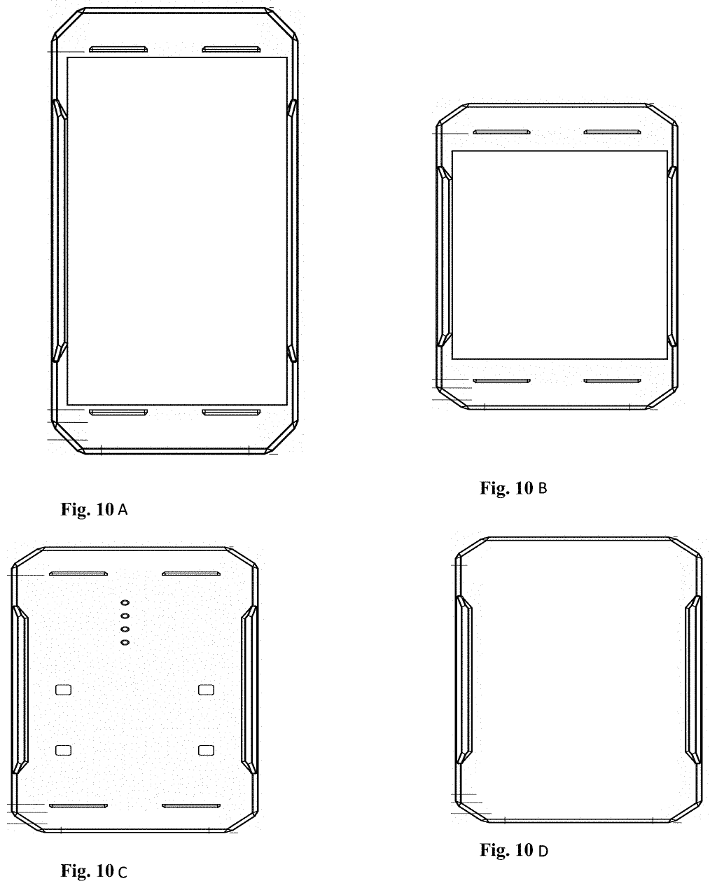

FIG. 10 provides a top diagrammatic representation of the ACTIVE CARD HOLDER viewed from above with an interchangeable identification card in place, (FIGS. 10A and 10B), retained in association with the active base by the card retainer ridges, wherein FIG. 10A, shows a rectangular base in which not all four sides are substantially equal in length, while FIGS. 10B, 10C and 10D show a rectangular base in which all four sides are substantially equal in length. The active card reader represents an embodiment of the device according to this invention which is operative within the system and according to the method of this invention as disclosed herein and in U.S. Ser. No. 29/683,010, filed on Mar. 10, 2019, the contents of which are herein incorporated by reference.

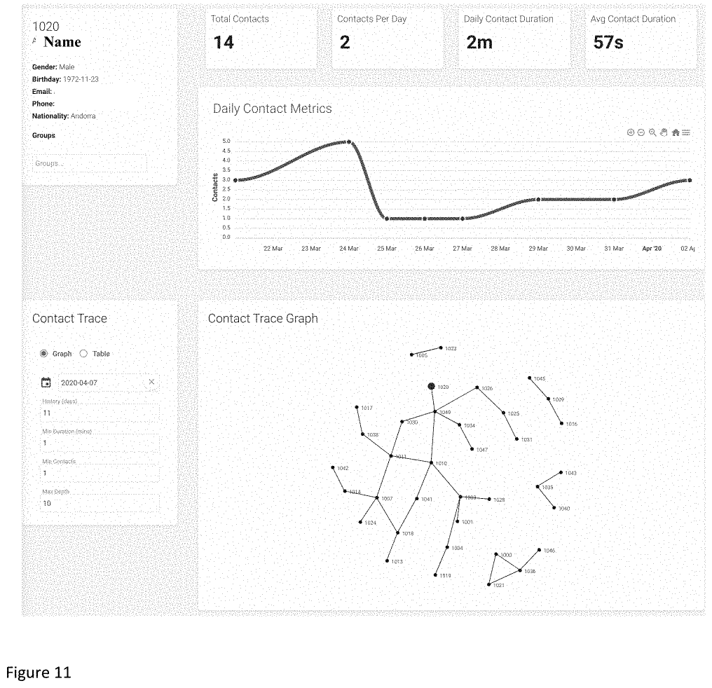

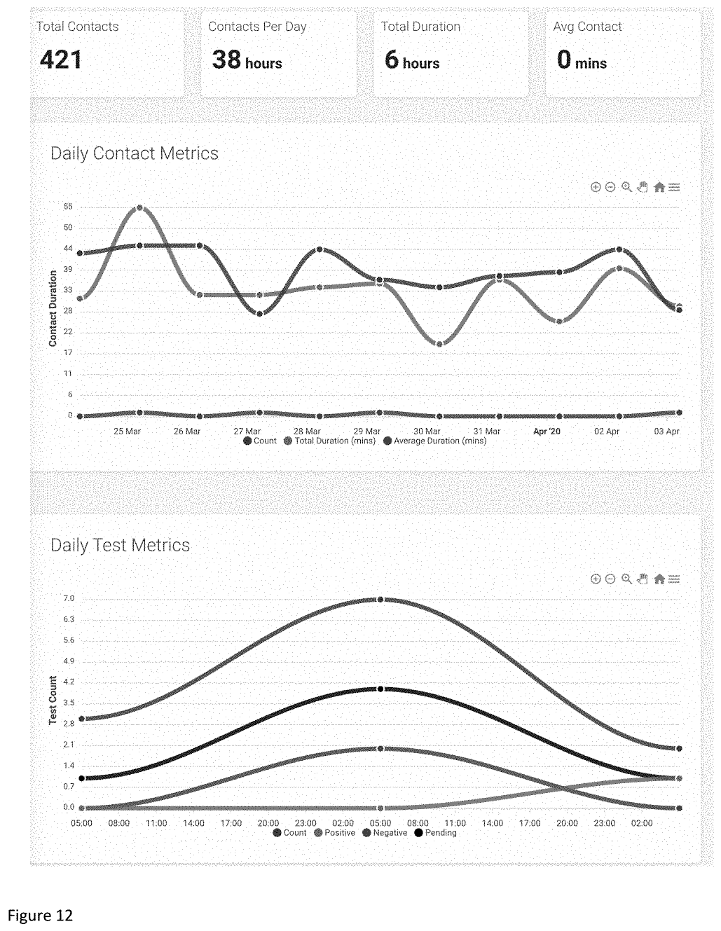

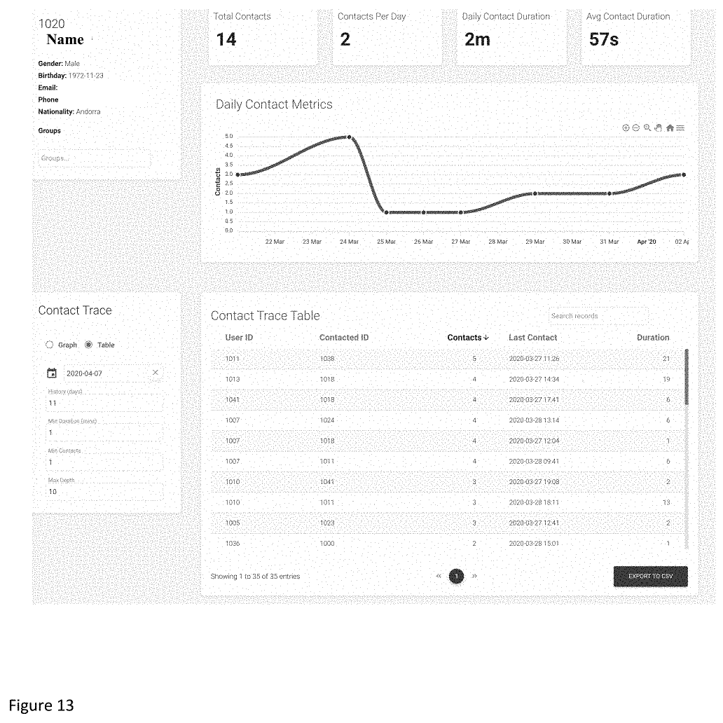

FIG. 11 provides a representation of data captured according to the system of this invention in which daily contact metrics are captured, including the total number of contacts captured, the number of contacts per day, the duration of contacts and the average contact duration, as well as a contact trace graph. FIG. 12 provides a representation of data captured according to the system of this invention in which daily contact metrics are captured, including the total number of contacts captured, the number of contacts per day, the duration of contacts and the average contact duration, along with daily test metrics. FIG. 13 provides a representation of data captured according to the system of this invention in which daily contact metrics are captured, including the total number of contacts captured, the number of contacts per day, the duration of contacts and the average contact duration, along with a table showing contacts. Each of these representations shows data captured according to an embodiment of this invention in which contract data is traced, captured, output for analytics and the analyzed data displayed to enable corrective action, asset tracking and the like.

Those skilled in the art are well aware of communication protocols between devices and, based on the present disclosure, are enabled to utilize the device, system and method of this invention to track assets and people and interactions between them. The Internet of Things (“IOT”) is now all-pervasive in the world and more and more devices are wi-fi enabled or can communicate with wi-fi enabled or cellular devices which can tap into Global Positioning Systems (“GPS”) which are already in existence, to provide location acuity.

In one embodiment, the device is enabled with known RFID or Bluetooth®, NFC, or a combination thereof, or similar short-range, low power, communication technology protocols and hardware. Such a device, while unable to properly map precise location via GPS on its own, may provide precise location via short range communication with static reference beacons within an enclosed or constrained space, or the location acuity is provided by immediate upload from the proximity sensor device of this invention to a smartphone, a separate dedicated devices, or the like, including via, e.g. a gateway, such as that described further herein below.

To sense proximity within a pre-defined proximity setting, data is generated for encounters which transgress or trigger the proximity setting, the data is stored, and uploaded to e.g. a cell-phone, a computer, or dedicated hardware, which then does analytics to identify encounters that are potentially involved in disease vector transmission, misuse of assets or the like.

Contact tracing has been one of the great failings as the COVID19 pandemic has evolved which the present invention addresses, either in decentralized system or a centralized system Medical professionals are able to query data transmitted by the device of this invention in a centralized system, as it is transmitted in real time. Alternatives are periodic uploads of data whenever the device connects to a cell phone or wi-fi hotspot. In a preferred version of this embodiment, when two people are in such proximity as to violate the proximity threshold coded into the device, the device emits an alarm, which may be audible, visual, vibratory, or the like and combinations thereof.

5.1 Enabling Embodiments and Written Description as to How to Make and How to Use this Invention:

In one of its simplest embodiments, the device comprises a housing, such as a wristband, a pin, or the like, with an RFID or Bluetooth® chip capable of detecting and recording the proximity of like devices within a relatively short, pre-defined distance rage. In one embodiment, ability to set distance criteria is pre-defined in the hardware, while in another, such criteria are subject to modification by firmware updates or modifications that are user-controlled via a user interface on the device. The device, via the chipset chosen detects proximity of like devices, and stores data regarding the proximity event meeting pre-set proximity data capture specifications in an on-board memory component in more complex embodiments, the captured data is delivered to a central system by, either directly by on-board cellular or wi-fi communication chipsets, or by communicating with e.g. a paired cell phone which already has on-board cellular and wi-fi communication capabilities.

In a preferred embodiment according to this invention, the device is a BLE wristband which communicates with a BLE Gateway which receives data via Bluetooth® and sends the data, e.g. via GSM, to a web server. The robust BLE Gateway receives data from all sensors that transmit data via Bluetooth®. Therefore, it is possible to transmit the data of multiple sensors over one BLE Gateway. With Bluetooth® Smart (formerly BLE) ranges up to 20 m can be achieved. For a transmission, for example from a field or a canal to an office, a 20 m range may not be sufficient. In such an instance, a GSM BLE Gateway solves the problem and bridges the distance by roaming mobile data connection. Thus, the BLE Gateway offers permanent access to data.

In an alternate embodiment according to this invention, the device is an Active Card Holder wherein the device provides all of the communications technology as described herein, the ID card provides information on the individual to whom the Active Card Holder is issued, for interaction with the System according to this invention.

### 5.2 How the Proximity Sensing Operates:

For proximity sensing to occur, the required hardware required is a Bluetooth® radio module or equivalent radio module if alternate communications technologies are implemented in the device. Firmware is loaded onto the device to provide an interface to control the Bluetooth® radio module. Bluetooth® radio module are commercially available from manufacturers with the firmware provided pre-installed in the module's memory. The firmware controls the overall device logic, including, when it uses the Bluetooth® radio module, how it uses the Bluetooth® radio module, how it stores acquired data, and how/when it transmits data to other devices for further processing. Proximity settings are achieved in any of several ways, depending on the embodiment, including via: non-adjustable (“hard-coded”) settings as part of the overall device firmware; automatically adjusted settings, depending on pre-defined rules stored in device memory, adjustable settings via commands sent to the device via Bluetooth® or other means of communication with the device. This could take the form of: a mobile application with a Bluetooth® User Interface; a user Interface on a Web Application including via Cell Network; automatically adjusted based on pre-defined rules stored on server with which the device is in communication. By adjusting settings, the distance, frequency, and duration of proximity measurements is altered as required. The following settings are typically used:

### I. Emitting Device

a. Device Identifier—provides means to identify which device emitted a detected advertisement;
b. Device Transmission Power—determines strength of advertisement and therefore distance at which the advertisement can be detected,
c. Transmission Interval—determines how frequently the device emits an advertisement.

### II. Detecting Device

a. Scan Filters—used to filter out detected advertisements that do not match set criteria such as Sets of Device Identifiers and Minimum Signal Strength;
b. Scan Interval—determines how frequently the device scans for advertisements; and
c. Scan Duration—determines the length of each scan.

Upon detecting other devices, logic contained in the firmware preferably adjusts these settings automatically to optimize the process. Furthermore, logic rules are defined to trigger events when a specific device or quantity of devices is detected. An example of such logic with which the device of the present invention is enabled is as follows:
IF—an advertisement with a device identifier matching known devices is detected
THEN—store the unique device identifier, the time at which it was detected and the duration it was in detectable range

The collected data, in one embodiment, is stored on the device in numerous ways depending on use case/hardware configuration:
1. Random Access Memory
2. On-chip Memory
3. Flash Memory
4. SD Card
5. Various other means known in the art.

Once collected, the data is either retained onboard the device, or it is transmitted to another device according to any of the following ways:
1. Bluetooth®.
   a. Device to Device
   b. Device to Cell Phone
   c. Device to PC
   d. Device to Dedicated Bluetooth® Gateway to Server
2. WIFI
   a. Device to PC
   b. Device to Cell Phone
   c. Device to WIFI Gateway to Server
3. Cellular Network
   a. Device to Server
   b. Device to Cell Phone
4. USB or Equivalent Cable
   a. Device to PC
   b. Device to Cell Phone
      5.3 How the Device System and Method of this Invention Utilizes Bluetooth®

A device with a Bluetooth® radio emits Bluetooth® signals called “advertisements” at pre-determined intervals with a pre-determined transmission power. These advertisements consist of data that is encoded using sets of rules called “protocols”. These rules are used to enable communication between various Bluetooth® devices. Each protocol has its own rules about how data should be organized and may be considered analogous to a language used by humans to speak to each other. Each advertisement contains (but is not limited to) one or more of the following:
Device identifier
Device transmission power
Expected device signal strength obverse by another device when the two devices are 1 meter apart from each other
Device sensor data such as:
Device battery voltage
Count of advertisements emitted since being turned on
Temperature
Humidity
Various other sensor data depending on what modules are included in the device's electronic circuitry

The measured signal strength of an advertisement (often described in RSSI units) is proportional to the distance between the emitting device and the receiving device. The distance between devices can be roughly calculated using:
Measured signal strength of advertisement
Transmission power setting (contained in advertisement data)
Expected device signal strength (contained in advertisement data)

If only proximity detection (and not specific distance measurement) is required, the Transmission Power of each device is configured such that the device will only be detected by other devices within a certain range. The desired settings are determined empirically or by using TX Power vs Signal Strength plots provided by hardware manufacturers. A special protocol that is part of the Bluetooth® Low Energy (BLE) standard and common to most devices is called Generic Attribute Profile (GATT). The GATT protocol allows one device to interact with a second device by sending commands to (among other things) enable/disable device functionality, change configurable settings, and read data stored on the second device.

### 5.4 Embodiments and Features Thereof:

In preferred embodiments, Bluetooth® or other near-proximity device to device communications are used in the system to generate data reflecting monitored distance, proximity, time, and duration of proximity between people, equipment, and/or tools. The data from the Bluetooth® device is sent to a secondary device using WiFi, Bluetooth®, Cellular Networks, or other means of device to device communications. The data is utilized to calculate more advanced metrics that are useful and valuable to prevent disease transmission and appropriate asset tracking via configurable settings to set proximity measurement limits and detection intervals. The device transmits data in real-time or stores data on the device until it is ready to be transmitted. Preferably, the data is encrypted to protect the data, including who each person wearing the device is, who or what they interacted with, and for how long. Different physical embodiments, including shape, size, format and the like is optimized and adapted for a particular use while maintaining underlying functionality. Accordingly, different embodiments of the device include but are not limited to a bracelet, an identification badge, an asset tag, or the like.

Where the device is implemented in a system for contact tracing for disease transmission prevention, the data is analyzed to track whether, when, for how long and where an infected person has come into contact with an uninfected person. In a preferred version of this embodiment, when two people are in such proximity as to violate the proximity threshold coded into the device, the device emits an alarm, which may be audible, visual, vibratory, or the like and combinations thereof.

Where the device is implemented in a system for contact tracing for usage, productivity, cost, and efficiency analytics, the data is analyzed to calculate whether, when, for how long and where a given person has come into contact with a given asset and whether such contact is appropriate for that individual, that location, and for the measured duration. Where a contact between an individual and a tracked asset is found to be improper, the individual's device, in one preferred embodiment, emits an alarm, which may be audible, visual, vibratory, or the like and combinations thereof.

Accordingly, in light of the foregoing disclosure, those skilled in the art will appreciate that this invention includes a device having a housing, a means for near-proximity wireless communication to transmit data to a second device equipped to receive such data, identifying the owner of the device, and which receives data identifying the owner of any said second device or the identity of equipment with which said second device is associated, a means for sensing proximity of said second device and the identity of the owner thereof or the identity of equipment with which said second device is associated, which second device transmits data to any other like device equipped to receive such data, identifying the owner of said second device or the identity of equipment with which said second device is associated, a means for defining proximity criteria to be met for recording, as stored data, a contact event when a threshold proximity between said device and a second device occurs, a means for recording, as stored data, the time and duration of said contact event, optionally, means for recording as stored data the location of said contact event; and a means for storage of said data recording the occurrence of the contact event, the identity of the individuals or equipment involved in the contact event, time and duration of the contact event, and, if present, data recording the location of the contact event.

In a preferred embodiment, device is enabled to communicate the stored data to a second device. Elements of the device include a wireless communication antenna, a tag which wirelessly transmit a unique identity associated with an individual utilizing the device, a tag reader to read the identity of an individual utilizing a second such device or equipment tagged with a second such device, and a power source. Preferably, the device includes updateable firmware whereby a proximity threshold for recording a contact event is defined. Preferably, when a proximity threshold has been violated, the device provides an alarm which may be auditory, visual, vibratory, or a combination thereof.

In a further aspect, the invention is a system which utilizes a device as described herein above. Preferably, the system includes both a device as described herein above and at least one second device as described herein above. Preferably, the system includes a plurality of such devices. In such a system, preferably, data is transmitted by each device is received by a central processing unit which analyzes the data and identifies correlations between pathogen or disease spread and Contact Events measured by each device included in the system, or which outputs correlations between locations of people and assets each bearing said device, or sends out an alarm to each person bearing such a device to alert them to move away from another person who is too close to them.

In yet a further aspect, the invention is a method which includes providing a device as described herein above to a person whose activities and proximities to other people and assets is tracked. Preferably, tracking data is obtained as to the activities and proximities to other people and assets and the data is analyzed to provide real-time or retrospective acuity as to the proximities and contacts of a person with other persons and assets which themselves are provided or tagged with a device as described herein above.

In a particularly preferred embodiment according to the invention, the device consists essentially of (a) an audible, visual or vibratory alarm, normally in a non-activated state, and (b) near-proximity wireless communication hardware adapted for (i) transmission of data announcing its presence to any like device, and (ii) receipt of data announcing the presence of a like device, such that said alarm of said device is activated upon detection by said device of data announcing the presence of a like device within a defined proximity. A system according to this aspect of the invention comprises at least two such devices, and preferably, comprises a plurality of such devices. In this aspect of the invention, a method to prevent disease spread, for example, comprises providing such a device to a person which alerts that person when a person or asset bearing a like device according is detected within a defined or too near a proximity. In such a method, each person, upon being alerted, reestablishes their distance from other persons, and the alarm on the device reverts to its normally non-activated state. Such a system, method and device is inexpensive to produce and widely distribute, and, with global distribution thereof, with each new device added to the system, the system provides greater protection to all participating in the use of the system.

### 6.0 EXAMPLES

Having generally described this invention, including its preferred embodiments and how to make and use such embodiment, the following Examples are provided to further describe and enable this technology, using Bluetooth® Proximity technology, using both proximity and duration of time components, which enable advanced analytics as required to ensure that e.g. infected people are not coming into contact with non-infected people, or assets are being used by the correct people at the correct time and location for the correct duration of time.

### Example 1

### Equipment/Tool Proximity to Worker Used for Productivity Analysis

A Bluetooth® device is fixed to a piece of equipment or a tool such that the device emits an advertisement containing a unique identifier that is associated with the equipment/tool in the system according to this invention. A worker carries another Bluetooth® device that scans for nearby device advertisements at a set interval. The number of times the device carried by the worker detects the device affixed to the piece of equipment or the tool multiplied by the scan interval of the device carried by the worker is used to calculate the time the worker was in proximity to the piece of equipment or tool. This value is used to calculate equipment/tool usage by each worker which is aggregated into productivity analytics describing various equipment/tool metrics for the entire project.

### Example 2

Equipment/Tool/Worker Proximity to Equipment/Tool/Worker for Location Estimation A Bluetooth® device is fixed to a piece of equipment or a tool or carried by a worker. This device emits an advertisement containing a unique identifier that is associated with the equipment/tool/worker in the system of the invention. A second equipment/tool/worker is assigned another Bluetooth® device that scans for nearby device advertisements at a set interval. The second device also measures and reports its Location by a separate method such as GPS coordinates or WIFI SSID detection. The location reported by the second device is used to estimate the location of the first device since the second device's exact location is known and the first device is in close proximity to the second device or it would not be detecting it. This is valuable because device battery life can be significantly improved because the Bluetooth® Radio requires less energy than a GPS module or WIFI Radio, and financial cost of location measurement can be reduced by not having to rely on WIFI SSID detection methods which costs money per location attempt.

### Example 3

### Equipment/Tool/Worker Proximity to Fixed Device for Location Estimation

One or more Bluetooth® devices are fixed to immobile surfaces and their positions are recorded in the system of the invention. An equipment/tool/worker is assigned another Bluetooth® device that scans for nearby device advertisements at a set interval. The second device reports detected devices and RSSI values to the system. The system uses these measurements to calculate the position of the Bluetooth® device assigned to the equipment/tool/worker. This is valuable because device battery life can be significantly improved because the Bluetooth® Radio requires less energy than a GPS module or WIFI Radio, and the financial cost of location measurement can be reduced by not having to rely on WIFI SSID detection methods which cost money per location attempt.

### Example 4

### Worker to Worker Proximity for Fraud Detection

Where the device is used to measure productivity metrics, some workers may attempt to defraud the system to be credited for work which they did not perform. A method to detect attempted fraud wherein one worker carries multiple Bluetooth® may occur when one Bluetooth® device is assigned to one worker and a second Bluetooth® device is assigned to a second worker. If the first worker carries the first and second devices in an attempt to defraud the system, both devices scan for Bluetooth® advertisements at a pre-determined interval and report detected advertisements to the system. The system detects that these devices have been in proximity to each other for a significantly longer time than one would expect if the devices were being carried by two separate workers. The system then alerts users, managers, or the like that these workers may be engaging in fraud.

### Example 5

### Contact Tracing to Limit Disease Transmission

A Bluetooth® enabled device according to this invention is worn by a first and a second individual. The first individual is found to be infected with a communicable disease. When the first person and the second person come into unacceptably close proximity to each other so as to violate the proximity threshold coded into the device, the device emits an alarm, which may be audible, visual, vibratory, or the like and combinations thereof. The first individual, the second individual or both are alerted. Alternatively, where the disease status of the first individual, the second individual or both is not known at a first time, T1, but at a later time is confirmed to be infected, the system permits contact tracing to occur to identify every individual that has been in sufficiently close proximity, for a sufficiently long period of time with the infected individual can be traced and alerted to avoid contact with others until it is certain that they have not become infected or infectious. In a preferred embodiment according to this invention, without the need for any external monitoring or data uploading or analytics, the system comprises wearable wristbands which alerts the wearer that they are too close to another individual, and either or both individuals are alerted by a vibratory, optical or auditory alert so that appropriate social distancing is re-established. Of course, in such embodiment, additional data and analytics are available when the device stores or transmit the captured data to external devices for contact tracing and disease transmission containment.

### Example 6

### Additional Use-Cases for Embodiments of the Device, System and Method of this Invention

Those skilled in the art will appreciate that the invention disclosed herein, in various embodiments, is useful and applicable in at least the following use cases to provide data for analytics:
1. Use proximity of people and items combined with ambient BLE-WiFi signals to determine whether an item has been removed from a restricted area and generate an alert.
2. Use proximity of people and items combined with ambient BLE/WiFi signals to create spatiotemporal models that improve location accuracy of existing technologies.
3. Use proximity of people combined with location to model and improve traffic/resource flow through closed quarters environments.
4. Use proximity of people and items to model work productivity in closed quarters environments where efficiency decreases as population/sq. ft. increases
5. Use proximity of people and items to detect inefficiency by measuring how often people/items leave and re-join a cluster of people/items.
6. Use proximity of people to model team cohesion.
7. Use proximity of people and efficiency metrics to analyze impact of team composition on productivity/efficiency
8. Use of proximity of people with ambient BLE/WiFi signals to model disease transmission probabilities using neural networks and alert users if models suggest they are at higher risk.

### Example 7

### Sensors and Communication Hardware

In one embodiment according to the invention, in addition to the communication hardware described herein for proximity sensing, the device further includes sensors for measuring physiologic parameters, e.g. temperature, heart rate, blood pressure or the like, which metrics are utilized to provide an initial assessment of whether the bearer of the device may be ill and possibly infectious. In the same or another embodiment, the device includes e.g. GPS enabling hardware, WiFi connectivity hardware, cellular data communication capabilities, or combinations thereof. Whatever is stated herein for one embodiment is applicable to all embodiments, unless expressly excluded or unless, under the circumstances, it is evident that such features are not present in a given embodiment—e.g. a completely stand-alone and self-contained system where only the device is required to sense proximity of another device and to set of an alert.

What is claimed is:

1. A device comprising:
a. a housing;
b. means for near-proximity wireless communication to transmit data to a second device equipped to receive such data, identifying the owner of the device, and which receives data identifying the owner of any said second device or the identity of equipment with which said second device is associated;
c. means for sensing proximity of said second device and the identity of the owner thereof or the identity of equipment with which said second device is associated, which second device transmits data to any other like device equipped to receive such data, identifying the owner of said second device or the identity of equipment with which said second device is associated;
d. means for defining proximity criteria to be met for recording, as stored data, a contact event when a threshold proximity between said device and a second device occurs;
e. means for recording, as stored data, the time and duration of said contact event;
f. optionally, means for recording as stored data the location of said contact event; and
g. means for storage of said data recording the occurrence of the contact event, the identity of the individuals or equipment involved in the contact event, time and duration of the contact event, and, if present, data recording the location of the contact event.

2. The device according to claim 1, wherein said device communicates said stored data to a second device.

3. The device according to claim 1 comprising:
a. a wireless communication antenna;
b. a tag which wirelessly transmit a unique identity associated with an individual utilizing the device;
c. a tag reader to read the identity of an individual utilizing a second such device or equipment tagged with a second such device; and
d. a power source.

4. The device according to claim 3 further comprising updateable firmware whereby a proximity threshold for recording a contact event is defined.

5. The device according to claim 1 wherein, when a proximity threshold has been violated, the device provides an alarm.

6. The device according to claim 5 wherein said alarm is auditory, visual, vibratory, or a combination thereof.

7. A device according to claim 1 consisting essentially of (a) an audible, visual or vibratory alarm, normally in a non-activated state, and (b) near-proximity wireless communication hardware adapted for (i) transmission of data announcing its presence to any like device, and (ii) receipt of data announcing the presence of a like device, such that said alarm of said device is activated upon detection by said device of data announcing the presence of a like device within a defined proximity.

8. A system comprising at least two devices according to claim 1.

9. A system comprising a plurality of devices according to claim 1.

10. A system according to claim 8 wherein the data transmitted by each said device is received by a central processing unit which analyzes said data and outputs correlations between pathogen or disease spread and Contact Events measured by each device included in the system, or which outputs correlations between locations of people and assets each bearing said device.

11. A system comprising at least two devices according to claim 7.

12. A system comprising a plurality of devices according to claim 7.

13. A system according to claim 7 consisting essentially of a plurality of said devices.

14. A system according to claim 7 consisting of a plurality of said devices.

15. A method comprising providing a device according to claim 1 to a person whose activities and proximities to other people or assets is tracked.

16. The method according to claim 15 wherein tracking data is obtained as to the activities and proximities to other people and assets and said data is analyzed to provide real-time or retrospective acuity as to the proximities and contacts of a person with other persons or assets which themselves are provided or tagged with a device according to claim 1.

17. A method comprising providing a device according to claim 7 to a person which alerts that person when a person or asset bearing a like device according to claim 7 is detected within a defined proximity.

18. The method according to claim 17 further comprising each person upon being alerted reestablishes their distance from other persons such that the alarm on the device reverts to a its normally non-activated state.
